(19) United States
(12) Patent Application Publication
(10) Pub. No.: US 2023/0070998 A1
(43) Pub. Date: Mar. 9, 2023

(54) A METHOD OF PREPARING A HYBRID CAPSULE AND RELATED PRODUCTS

(71) Applicant: Agency for Science, Technology and Research, Singapore (SG)

(72) Inventors: Praveen Thoniyot, Singapore (SG); Alexander Maria Van Herk, Singapore (SG); Wenguang Zhao, Singapore (SG)

(21) Appl. No.: 17/285,302

(22) PCT Filed: Oct. 15, 2019
(86) PCT No.: PCT/SG2019/050511

§ 371 Date: Apr. 14, 2021

(30) Foreign Application Priority Data
Oct. 15, 2018 (SG) 10201809076S

(51) Int. Cl. B01J 13/22; A61K 8/11; A61K 8/25; A61K 8/81

(52) U.S. Cl. CPC B01J 13/22; A61K 8/11; A61K 8/25; A61K 8/8158; A61K 2800/412; A61K 2800/10; A61K 2800/624; A61K 2800/651

(57) ABSTRACT

There is provided a method of preparing a hybrid capsule, the method comprising heterocoagulating organic polymer latex particles with a primary capsule to form an organic polymer coating layer over a shell of the primary capsule.

### TECHNICAL FIELD

The present disclosure relates broadly to a method of preparing a hybrid capsule as well as a hybrid capsule prepared by said method.

### BACKGROUND

Capsules, particularly those in the micron or submicron size, that are capable of encapsulating a variety of actives offer great promise in a large number of consumer applications, including but not limited to perfumes, coatings, medicines, agricultural chemicals, catalysts, printings, films, fibers and cosmetics.

However, to date, the current technology used to produce such capsules suffers from several drawbacks. For example, available technology for producing flowable silica capsules for consumer care applications relies on the emulsification of an active in water using a neutral surfactant and growing silica shell around the micron sized droplet. Typically, the silica capsules obtained through such methods are easily breakable under mechanical stress and cannot withstand the high shear that is needed for many applications. Furthermore, the silica capsules obtained through such methods are also highly porous, making them unsuitable for applications where hermetic sealing is desired, for example in perfumes.

Accordingly, attempts were made to produce reinforced capsules. Some of the techniques that aim to reinforce capsules include (i) layer by layer coating; (ii) grafting from the surface of capsules; (iii) infiltration of polymer electrolyte and cross linking; (iv) surface functionalization and polymerization; and (v) precipitation polymerization. However, these techniques are usually costly, complicated, complex and/or require long processing time. This is because many of these techniques involve a large number of different types of chemical reactants, reaction steps, reaction conditions and post-coating processing steps. As a result, such techniques also have limited scalability.

In addition, these techniques create new problems. For example, many of the chemical reactants used or by-products produced are toxic to the environment, therefore creating a disposal problem. Furthermore, the high chemical reactivity of several of the chemical reactants used or by-products produced makes such techniques incompatible for use with chemically sensitive actives as well as for consumer care applications, where consumer safety is of paramount importance.

Therefore, there is a need for a method and a capsule that overcome or at least ameliorate one or more of the problems discussed above.

### SUMMARY

According to one aspect, there is provided a method of preparing a hybrid capsule, the method comprising heterocoagulating organic polymer latex particles with a primary capsule to form an organic polymer coating layer over a shell of the primary capsule.

In one embodiment, the hybrid capsule is an organic-inorganic capsule and the primary capsule is an inorganic capsule.

In one embodiment, the heterocoagulating step is at least partly carried out at a temperature that is no less than the glass transition temperature (Tg) of the organic polymer latex particles.

In one embodiment, the heterocoagulating step is at least partly carried out at a temperature of from 10° C. to 80° C. and/or at a pH from 2 to 11.

In one embodiment, the heterocoagulating step is carried out in the presence of two opposing charges, a first charge being associated with the organic polymer latex particles and a second charge being associated with the primary capsule, the second charge having a polarity that is opposite to that of the first charge.

In one embodiment, the method further comprises introducing the primary capsule into a larger volume of the polymer latex particles prior to the heterocoagulating step.

In one embodiment, the step of introducing the primary capsule comprises introducing a plurality of primary capsules until the concentration of the primary capsules in the mixture of primary capsules and organic polymer latex is from 10% to 40% by weight of the entire mixture.

In one embodiment, the heterocoagulating step to form a polymer coating layer over a shell of the primary capsule is substantially devoid of a polymerization reaction.

In one embodiment, the heterocoagulating step is carried out in the absence of an organic solvent.

In one embodiment, the organic polymer latex particles comprise poly-N-isopropyl acrylamide (PNIPAM) latex particles, poly-methyl-methacrylate-co-poly-styrene-co-polyethyl-hexyl-acrylate-co-poly-acrylic acid latex particles, poly caprolactone (PCL) latex particles, poly valerolactone latex particles, poly butyrolactone latex particles, polyurethane latex particles, polyamide latex particles, polyacrylic acid-containing latex particles or combinations thereof.

In one embodiment, the primary capsule comprises a silica capsule, a zirconia capsule, a titania capsule or combinations thereof.

In one embodiment, the polymer latex particles have an average particle size in the range of from 50 nm to 1000 nm and the primary capsule has an average particle size in the range of from 1 μm to 100 μm.

In one embodiment, the heterocoagulating step is carried out in the presence of at least two different surfactants comprising at least one cationic surfactant and at least one anionic surfactant.

In one embodiment, the at least two different surfactants are independently selected from the group consisting of a primary amine surfactant, a secondary amine surfactant, a tertiary amine surfactant, a quaternary amine surfactant, cetyl trimethylammonium bromide (CTAB), sodium dodecyl sulfate (SDS), carboxylic acid salt, sulfonic acid salt, phosphoric acid ester, alcohol sulfate, alkylbenzene sulfonate or combinations thereof.

According to another aspect, there is provided a hybrid capsule comprising a primary capsule having a shell; and an organic polymer coating layer over the shell of the primary capsule.

In one embodiment, the hybrid capsule is an organic-inorganic capsule and the primary capsule is an inorganic capsule.

In one embodiment, the shell of the primary capsule is substantially hermetically sealed by the polymer coating layer.

In one embodiment, the hybrid capsule is micron- or submicron-sized.

In one embodiment, the hybrid capsule is substantially resistant to breaking under scanning electron microscopy (SEM) vacuum conditions.

In one embodiment, the hybrid capsule comprises one or more actives loaded in a core of the primary capsule that is encapsulated by the shell of the primary capsule.

In one embodiment, the organic polymer coating comprises poly-N-isopropyl acrylamide (PNIPAM), poly-methyl-methacrylate-co-poly-styrene-co-polyethyl-hexyl-acrylate-co-poly-acrylic acid, poly caprolactone (PCL), poly valerolactone, poly butyrolactone, polyurethane, polyamide, polyacrylic acid or combinations thereof and the primary capsule comprises a silica capsule, a zirconia capsule, a titania capsule or combinations thereof.

In one embodiment, the hybrid capsule is configured to release at least a portion of one or more actives from the core when stimulated by a change in one or more of a salt concentration, a pH, a temperature or a mechanical pressure.

### DEFINITIONS

The term “latex” as used herein is to be interpreted broadly to refer to any dispersion/emulsion of one or more polymer(s) and/or copolymer(s).

The terms “organic-inorganic” and “inorganic-organic” as used herein represent the types of components present and the terms may be interchangeably used. Accordingly, these terms indicate the presence of an organic component and an inorganic component. In most situations, these terms are not intended to represent the positional relationship between the components unless otherwise stated or required by the context.

The term “micro” as used herein is to be interpreted broadly to include dimensions from about 1 micron to about 1000 microns.

The term “nano” as used herein is to be interpreted broadly to include dimensions less than about 1000 nm.

The term “particle” as used herein broadly refers to a discrete entity or a discrete body. The particle described herein can include an organic, an inorganic, a hybrid or a biological particle etc. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Example, non-limiting embodiments of a method of preparing a hybrid capsule, and said hybrid capsule are disclosed hereinafter.

In various embodiments, there is provided a method of preparing a hybrid capsule. In various embodiments, the capsule comprises an outer shell encapsulating an inner core that is capable of carrying one or more cargoes/actives. The core may be empty or loaded with one or more cargoes/actives. Accordingly, in various embodiments, the capsule is different from a solid particle that does not have a core for carrying one or more cargoes/actives.

The hybrid capsule may be a composite capsule. In various embodiments, the hybrid capsule comprises at least two different materials in its composition and/or parts of its structure. For example, the hybrid capsule may comprise a coating over a shell where the shell is formed of a different material from the coating. The different materials may be different in that the chemical make-up in each material is different. The chemical make-up in each material may be different in terms of the identity of the elements present and/or the molar ratio of the elements present and/or chemical arrangement of the elements. For example, the elements present in each material may be the same but the molar ratio and/or chemical structure in each material may be different and therefore these materials are still considered non-identical materials. In various embodiments, the hybrid capsule comprises an organic-inorganic capsule or an inorganic-organic capsule.

In various embodiments, the method comprises heterocoagulating organic polymer latex particles with one or more (or at least one) primary capsule(s) to form an organic polymer coating layer over a shell of the primary capsule. In various embodiments, the polymer coating layer is on top of or is on an outer/external surface of the shell of the primary capsule such that the shell is positioned between the polymer coating layer and an inner core of the primary capsule. The polymer coating may be in direct contact with the shell of the primary capsule, for example, the polymer coating may be disposed on the shell of the primary capsule.

In various embodiments, heterocoagulating the organic polymer latex particles with the shell of the one or more primary capsule is carried out such that the organic polymer latex particles are converted into a continuous film/coating on the surface of the shell of the primary capsule to form the hybrid capsule (e.g. an organic-inorganic capsule in some examples). In various embodiments, the heterocoagulating step comprises adsorbing the organic polymer latex particles onto the shell of the at least one primary capsule; coalescing the organic polymer latex particles to form polymer strands; and allowing the polymer strands to interpenetrate/inter diffuse with the shell of the primary capsule to form an organic coating on the shell for example such that a hybrid shell (e.g. an organic-inorganic shell in some examples) is obtained. The adsorption, coalescence and/or interpenetration/inter diffusion may result in the pores of the shell of the primary capsule being substantially or fully blocked or result in the shell being substantially or fully hermetically sealed. The adsorption, coalescence and/or interpenetration/inter diffusion may also result in the organic polymer latex particles losing their substantially spherical structure. In various embodiments, the heterocoagulating step comprises physical changes to the organic polymer latex particles but does not comprise chemical changes to the organic polymer latex particles.

In various embodiments, the heterocoagulating step to form a polymer coating layer over a shell of the primary capsule is substantially devoid of a polymerization reaction. Accordingly, in various embodiments, the heterocoagulating step is devoid of the use of monomers or the participation of monomers in a polymerization reaction to form a polymer over the shell of the primary capsule. On the contrary, in various embodiments, preformed polymers instead of monomers are used. In embodiments of the method, the polymers are also responsive and/or degradable. Advantageously, embodiments of the methods disclosed herein are simple and straightforward (due to the use of preformed polymers) as compared to methods which require polymerization of monomers to form a polymer coating layer. Furthermore, as the coating reinforcement is carried out using physical or non-chemical methods (c.f. with polymerization) in embodiments of the methods disclosed herein, the inorganic capsules are not unnecessarily subjected to harsh chemical conditions which may compromise their integrity (for example, the capsules may collapse or break), potentially leading to leakages of any cargoes/actives contained in the capsules (e.g. the primary capsules). In various embodiments, the heterocoagulating step and/or the entire method is carried out in the absence of an organic solvent or medium. In various embodiments, the heterocoagulating step and/or the entire method is carried out using an aqueous medium such as water, optionally distilled water, as the primary medium. Advantageously, embodiments of the methods disclosed herein are safe and non-toxic to the environment as compared to methods which require the use of harsh and toxic chemicals and/or organic solvents.

Therefore, in various embodiments, the polymer coating is formed over the shell of the primary capsule in a physical or non-chemical manner Accordingly, in various embodiments, the method does not comprise chemically modifying/functionalizing/coupling/grafting the organic polymer latex particles or the shell of the primary capsule or their surfaces thereof to facilitate an integration of the organic polymer latex particles on or with the shell of the primary capsule. In various embodiments, the method does not comprise providing/adding a chemical curing/cross-linking agent (e.g. glutaraldehyde) or chemically curing/cross-linking the organic polymer latex particles to facilitate an integration of the organic polymer latex particles on or with the shell of the primary capsule. In various embodiments, the method does not comprise chemically/covalently attaching/conjugating/coupling/anchoring/grafting a functional group e.g. an amine group to the organic polymer latex particles or the shell of the primary capsule or their surfaces thereof to facilitate an integration of the organic polymer latex particles on or with the shell of the primary capsule. For example, in various embodiments, the method does not comprise using silane coupling agent such as γ-methacryloxypropyl trimethoxysilane in conjunction with a silica precursor such as tetraethyl orthosilicate (TEOS) to facilitate an integration of an organic material on or with a shell of the primary capsule. In addition, in various embodiments, the method does not comprise providing a preformed commercially available functional polymer such as lupranate (polymeric isocyanate and/or diisocyanate) to facilitate an integration of an organic material on or with a shell of the primary capsule.

In various embodiments, the method does not require providing a block co-polymer such as one derived from poly(2-(dimethylamino)ethyl methacrylate) (PDMA) and poly(benzyl methacrylate) (PBzMA). Such co-polymer are expensive and require many synthesis steps. Additionally, in various embodiments, the method does not rely on coacervation to facilitate an integration of an organic material on or with a shell of the primary capsule.

Furthermore, in various embodiments, the method does not rely on controlled radical polymerization such as surface-initiated atom transfer radical polymerization (ATRP), in situ polymerization and/or precipitation polymerization to facilitate an integration of an organic material on or with a shell of the primary capsule.

In various embodiments, the heterocoagulating step does not comprise heterocoagulating a monomer with the shell of the at least one primary capsule. For example, in various embodiments, the method does not comprise precipitating organo-alkoxysilane monomer or vinyl monomer around a silica core/capsule.

In various embodiments, the heterocoagulating step is at least partly carried out at a temperature that is within or close to, no less than, or higher than the glass transition temperature (Tg) range or melting temperature range of the organic polymer latex particles or the organic polymer from which the organic polymer latex particles are derived. Advantageously, this may aid the coalescence or merging or melting of the organic polymer latex particles to form a continuous film/coating over the surface of the shell of the primary capsule. Depending on the identity of the organic polymer latex particles used, the temperature applied to aid the coalescence or merging of the organic polymer latex particles may vary. Accordingly, a suitable temperature range may be used. For example, the heterocoagulating step may be carried out at a temperature of from about 10° C. to about 80° C., from about 15° C. to about 75° C., from about 20° C. to about 70° C., or about 10° C., about 15° C., about 20° C., about 25° C., about 30° C., about 35° C., about 40° C., about 45° C., about 50° C., about 55° C., about 60° C., about 65° C., about 70° C., about 75° C. or about 80° C.

Similarly, the heterocoagulating step may at least partly be carried out at a pH that can facilitate the coalescence or merging of the organic polymer latex particles for the formation of the coating layer over the shell of the primary capsule. Depending on the identity of the organic polymer latex particles used, the pH applied to aid the coalescence or merging of the organic polymer latex particles may vary. Accordingly, a suitable pH range may be used. For example, the heterocoagulating step may be carried out at a pH of from about 2 to about 11, from about 2.5 to about 10.5, from about 3 to about 10, from about 3.5 to about 10 or about 2, about 2.5, about 3, about 3.5, about 4, about 4.5, about 5, about 5.5, about 6, about 6.5, about 7, about 7.5, about 8, about 8.5, about 9, about 9.5, about 10, about 10.5 or about 11.

In various embodiments, the method and/or the heterocoagulation step further comprises physically curing the organic polymer latex particles. For example, the curing may be carried out by using at least one of temperature or pH as a curing means. In various embodiments, curing the organic polymer latex particles does not comprise chemically curing/cross-linking the organic polymer latex particles.

Accordingly, the method and/or heterocoagulating step may therefore also comprise one or more of: changing/adjusting/regulating a pH of the mixture, changing/adjusting/regulating a temperature of the mixture, or curing the organic polymer latex particles in the mixture optionally at room/ambient temperature or below room/ambient temperature. For example, this may include raising/increasing the pH of the mixture optionally by about 5 points, about 5.5 points, about 6 points, about 6.5 points, about 7 points, about 7.5 points, about 8 points, about 8.5 points or about 9 points. The changing/adjusting/regulating a pH of the mixture and/or curing the organic polymer latex particles in the mixture may comprise raising/increasing the pH of the mixture from about 2 to about 11, from about 2.5 to about 11, from about 3 to about 11, from about 3.5 to about 11, from about 4 to about 11, from about 2 to about 10.5, from about 2.5 to about 10.5, from about 3 to about 10.5, from about 3.5 to about 10.5, from about 4 to about 10.5, from about 2 to about 10, from about 2.5 to about 10, from about 3 to about 10, from about 3.5 to about 10, from about 4 to about 10, from about 2 to about 9.5, from about 2.5 to about 9.5, from about 3 to about 9.5, from about 3.5 to about 9.5, from about 4 to about 9.5, from about 2 to about 9, from about 2.5 to about 9, from about 3 to about 9, from about 3.5 to about 9, from about 4 to about 9.

Likewise, in various embodiments, changing/adjusting/regulating a temperature of the mixture and/or curing the organic polymer latex particles in the mixture comprises raising the temperature of the mixture optionally by about 70° C., about 65° C., about 60° C., about 55° C., about 50° C., about 45° C., about 40° C., about 35° C. or about 30° C. For example, the changing/adjusting/regulating a temperature of the mixture and/or curing the organic polymer latex particles in the mixture may comprise raising the temperature of the mixture from a first temperature of no less than about 10° C. to a second temperature of no more than about 80° C., from a first temperature of no less than about 15° C. to a second temperature of no more than about 80° C., from a first temperature of no less than about 20° C. to a second temperature of no more than about 80° C., from a first temperature of no less than about 25° C. to a second temperature of no more than about 80° C., from a first temperature of no less than about 30° C. to a second temperature of no more than about 80° C., from a first temperature of no less than about 10° C. to a second temperature of no more than about 75° C., from a first temperature of no less than about 15° C. to a second temperature of no more than about 75° C., from a first temperature of no less than about 20° C. to a second temperature of no more than about 75° C., from a first temperature of no less than about 25° C. to a second temperature of no more than about 75° C., from a first temperature of no less than about 30° C. to a second temperature of no more than about 75° C., from a first temperature of no less than about 10° C. to a second temperature of no more than about 70° C., from a first temperature of no less than about 15° C. to a second temperature of no more than about 70° C., from a first temperature of no less than about 20° C. to a second temperature of no more than about 70° C., from a first temperature of no less than about 25° C. to a second temperature of no more than about 70° C., from a first temperature of no less than about 30° C. to a second temperature of no more than about 70° C., from a first temperature of no less than about 10° C. to a second temperature of no more than about 65° C., from a first temperature of no less than about 15° C. to a second temperature of no more than about 65° C., from a first temperature of no less than about 20° C. to a second temperature of no more than about 65° C., from a first temperature of no less than about 25° C. to a second temperature of no more than about 65° C., from a first temperature of no less than about 30° C. to a second temperature of no more than about 65° C., from a first temperature of no less than about 10° C. to a second temperature of no more than about 60° C., from a first temperature of no less than about 15° C. to a second temperature of no more than about 60° C., from a first temperature of no less than about 20° C. to a second temperature of no more than about 60° C., from a first temperature of no less than about 25° C. to a second temperature of no more than about 60° C. or from a first temperature of no less than about 30° C. to a second temperature of no more than about 60° C.

In various embodiments, the method also comprises providing a mixture (or suspension/dispersion) comprising the organic polymer latex particles and the at least one primary capsule prior to the heterocoagulating step. The mixture may be provided as a mixture in an aqueous medium such as water. Accordingly, the organic polymer latex particles may be dispersed in an aqueous medium and the primary capsule may also be dispersed in an aqueous medium such that after they are mixed to form a mixture, the mixture will still be in an aqueous medium. Advantageously, because the aqueous medium is not an organic medium that is toxic, the method may be an environmentally friendly method.

In various embodiments, the method does not comprise providing substances such as free radicals that react or will potentially react with the cargo/active and cause or potentially cause the cargo/active to undergo chemical transformation to an undesired form. Accordingly, the polymer latex particles mixture, the primary capsule mixture and/or the mixture of polymer latex particles and primary capsules is/are substantially devoid of substances such as free radicals that react with the active and cause the active to undergo chemical transformation to an undesired form.

In various embodiments, providing a mixture (or suspension/dispersion) comprising the organic polymer latex particles and the at least one primary capsule is achieved by introducing the at least one primary capsule into a volume of the polymer latex particles prior to the heterocoagulating step. Accordingly, in various embodiments, the method further comprises a step of adding, in a controlled manner, the at least one primary capsule to the organic polymer latex particles prior to the providing step, not vice versa. In various embodiments, the method does not comprise a step of adding the organic polymer latex particles to the at least one primary capsule. For example, the primary capsules are added bit by bit into a pool of polymer latex particles instead of the other way around. Therefore, the step of adding the at least one primary capsule to the organic polymer latex particles may comprise adding a smaller volume of primary capsules to a larger volume of organic polymer latex particles. Without being bound by theory, it is believed that the manner of adding the primary capsule to the polymer latex particles may better allow for the primary capsule to be sufficiently surrounded by polymer latex particles so that the formation of the polymer coating layer may completely and continuously surround the shell of the primary capsule.

The method may further comprise a step of adding a second and subsequent primary capsules until the concentration of the primary capsules in the mixture reaches an appropriate range. Without being bound by theory, it is believed that if the concentration of the added primary capsules in the mixture is too high, the polymer latex particles may not sufficiently surround most if not all of the primary capsules to ensure that the subsequent formation of the polymer coating layer may completely and continuously surround the majority of each of the shells of the primary capsules. Accordingly, in various embodiments, the step of adding a second and subsequent primary capsules comprises adding a second and subsequent primary capsules until the concentration of the primary capsules in the mixture is from about 10% to about 40% by weight, from about 15% to about 35% by weight, from about 20% to about 30% by weight, or about 10% by weight, about 15% by weight, about 20% by weight, about 25% by weight, about 30% by weight, about 35% by weight or about 40% by weight.

In various embodiments, the method further comprises a step of mixing the organic polymer latex particles and the primary capsule(s) e.g. at no more than about 25 revolutions per minute (rpm), no more than about 24 rpm, no more than about 23 rpm, no more than about 22 rpm, no more than about 21 rpm, no more than about 20 rpm, no more than about 19 rpm, no more than about 18 rpm, no more than about 17 rpm, no more than about 16 rpm or no more than about 15 rpm (i.e. not high shear mixing), for example, in a 1 L reactor with stirring blades having smooth edges. The mixing may also be carried out at a different stirring speed in a different volume reactor (e.g. lab scale vs plant scale) and/or with different stirring blades (e.g. blade size) as those described above such that the shearing force/centrifugal force that exists during stirring is comparable, equivalent or similar to that produced using the above described parameters. The actual stirring speed used may also depend on factors such as the capsule type (hence capsule fragility) and the presence of stabilizing neutral surfactants in the system. Without being bound by theory, it is believed that an appropriate stirring speed may avoid aggregation of the primary capsules and facilitate the majority of each of the primary capsules to be properly surrounded by polymer latex particles so that the subsequent formation of the polymer coating layer may completely and continuously surround the majority of each of the shells of the primary capsules. The appropriate stirring speed may also preserve the integrity of the primary capsule(s).

In various embodiments, the method and/or the heterocoagulation step is carried out in the presence of different/opposite/opposing charges (e.g. opposite polarities and/or opposing polarities). For example, a first charge may be associated with the polymer latex particles and a second charge may be associated with the primary capsule. The first charge and the second charge may be opposite and/or having opposing polarities (e.g. one is a positive charge and the other is a negative charge) such that the opposite charges and/or opposing polarities can aid in bringing the organic polymer latex particles and the primary capsules together so as to allow individual primary capsules to be sufficiently surrounded by the organic polymer latex particles. That is, the different/opposite charges may promote attraction and enable coverage of the capsule surface with the latex particles. The first charge may be associated with the polymer latex particles by virtue of the charges that are present on the polymer latex particles per se (e.g. surface charge) or via the use of an intermediate such as a charged surfactant that surrounds the polymer latex particles. Likewise, the second charge may be associated with the primary capsules by virtue of the charges that are present on the primary capsules per se (e.g. surface charge) or via the use of an intermediate such as a charged surfactant that surrounds the primary capsules. Therefore, in some embodiments, the latex particles and primary capsules are surrounded by oppositely charged surfactants (e.g. one surfactant has a positive charge and the other surfactant has a negative charge) before they are being mixed together. It is also possible that if the latex particles inherently have a first charge on their surface (e.g. not by virtue of a surfactant), the surfactant used to stabilize the latex particles may be neutral (or not present at all if the latex particles are already sufficiently stable on their own) and the surfactant used to stabilize the primary capsules may have a second charge that is opposite to the first charge (or the surfactant may be neutral if the primary capsules already have the inherent second charge or the surfactant may not be present at all if the primary capsules already have the inherent second charge and are stable on their own) or vice versa. As an illustration, in a particular example, when the latex particles comprise NIPAM (poly-N-isopropyl acrylamide) and an acid such as acrylic acid, the acrylic acid in the latex gives a negative charge to the latex particles and so these surface charged polymer latex particles may be surfactant-free (e.g. any surfactant present may be removed by washing) before they are coated onto positively charged CTAB (cetyl trimethylammonium bromide) stabilized silica primary capsules.

Therefore, the method may also further comprise providing a surfactant to stabilize the organic polymer latex particles and a surfactant to stabilize the at least one primary capsule. These surfactants may be the same or different. In various embodiments, these surfactants are different. Accordingly, in various embodiments, the heterocoagulating step is carried out in the presence of at least two different surfactants. The surfactants may comprise charged surfactants. In various embodiments, the heterocoagulation is carried out in the presence of two different surfactants having charges that are opposite to each other. In various embodiments, the heterocoagulation is carried out in the presence of at least one cationic surfactant and at least one anionic surfactant. For example, in various embodiments, the charged surfactant that stabilises the primary capsule(s) has a charge that is opposite to that of the surfactant that is used to stabilize the organic polymer latex particles. Without being bound by theory, it is believed that synergistic interaction between oppositely charged surfactants (e.g. between a cationic surfactant and an anionic surfactant) favourably promotes heterocoagulation of the organic polymer latex particles with the primary capsule(s). When the oppositely charged surfactants present in each of the organic polymer latex particles dispersion and the primary capsules dispersion are mixed together (e.g. when both dispersions are mixed together), the opposite charges may attract and this may help to blend/integrate the organic polymer latex particles and the primary capsules so as to allow individual primary capsules to be sufficiently surrounded by the organic polymer latex particles. Therefore, the surfactants may be added to the dispersion of organic polymer latex particles and the dispersion of primary capsules before both dispersions are mixed together. Additional surfactants may also be added to impart extra stability to the hybrid capsules after they are formed e.g. after heterocoagulation. Each of the surfactants may independently be a cationic surfactant, for example the cationic surfactant may be one that is based on primary, secondary, or tertiary amines or quaternary ammonium cations, or the cationic surfactant cetyl trimethylammonium bromide (CTAB). Each of the surfactants may independently also be an anionic surfactant, for example the anionic surfactant may be one that is selected from sodium dodecyl sulfate (SDS), carboxylic acid salt, sulfonic acid salt, phosphoric acid ester, alcohol sulfate, alkylbenzene sulfonate or combinations thereof.

In various embodiments, the concentration of the surfactant for stabilizing the organic polymer latex particles dispersion is similar to the surfactant concentration used in emulsion polymerization, optionally wherein the concentration of the surfactant is much higher than the critical micellar concentration and/or concentration of the primary capsule and is from about 0.25% to about 2.5%, from about 0.50% to about 2.25%, from about 0.75% to about 2%, from about 1% to about 1.75%, or about 0.25%, about 0.50%, about 0.75%, about 1%, about 1.25%, about 1.50%, about 1.75%, about 2%, about 2.25% or about 2.5% by weight of the polymer latex particles. The surfactant may be completely or partially removed prior to heterocoagulation.

In various embodiments, the concentration of the surfactant for stabilizing the primary capsules dispersion is from about 0.5 to about 2.5% by weight of the primary capsules while initially making primary capsules. However, free surfactant may be removed completely or partially e.g. by washing prior to heterocoagulation. Hence, in various embodiments, the concentration of the surfactant for stabilizing the primary capsules dispersion is from about 0.1 to about 2% by weight during heterocoagulation.

In various embodiments, to reduce the likelihood of aggregation of differently charged surfactants, the method comprises using a substantially exact/accurate amount of surfactant needed by e.g. .theoretically calculating the surface area of the capsule b) removing extra surfactants e.g. by centrifugation, creaming or other approaches or c) using a neutral surfactant or a combination of the above.

The organic polymer latex particles may comprise at least one of synthetic latex, natural latex or combinations thereof. The polymer may be selected such that it is one that would allow inter-diffusion (e.g. between the inorganic shell) under aqueous conditions. In various embodiments, the organic polymer latex particles comprise water-dispersed organic polymer latex particles (no organic solvent). In various embodiments, the organic polymer latex particles comprise degradable polymer latex particles, biodegradable polymer latex particles, non-toxic polymer latex particles, bioresorbable polymer latex particles, responsive polymer latex particles, stimuli-responsive polymer latex particles, thermo-responsive polymer latex particles, thermoplastic polymer latex particles, poly-N-isopropyl acrylamide (PNIPAM) latex particles, poly-methyl-methacrylate-co-poly-styrene-co-polyethyl-hexyl-acrylate-co-poly-acrylic acid latex particles, poly caprolactone (PCL) latex particles, poly valerolactone latex particles, poly butyrolactone latex particles, polyurethane latex particles, polyamide latex particles, polyacrylic acid-containing latex particles or combinations thereof. Advantageously, embodiments of the method produce capsules that are responsive to one or more stimuli that may be useful in stimuli-driven delivery applications.

The organic polymer latex particles may optionally comprise a cross-linker optionally selected from bis-acrylates or acrylamides containing alkyl group or poly-ethylene group, or N,N′-Methylenebis(acrylamide) (BIS). In various embodiments, the heterocoagulating step is substantially devoid of the use/need of a cross-linker to form the organic polymer layer over the primary capsule. In some embodiments, where the organic polymer latex particles comprise poly caprolactone (PCL), the heterocoagulating step is carried out at a temperature of from about 60° C. to about 70° C., preferably 65° C., and/or without a cross-linker. In various embodiments, the organic polymer latex particles comprise no more than about 2% BIS, no more than about 1.5% BIS, no more than about 1.4% BIS, no more than about 1.3% BIS, no more than about 1.2% BIS, no more than about 1.1% BIS, no more than about 1.0% BIS, no more than about 0.9% BIS, no more than about 0.8% BIS, no more than about 0.7% BIS, no more than about 0.6% BIS, no more than about 0.5% BIS, no more than about 0.4% BIS, no more than about 0.3% BIS, no more than about 0.2% BIS or no more than about 0.1% BIS.

In various embodiments, the organic polymer latex particle is smaller in size/diameter/volume than the primary capsule. For example, the organic polymer latex particles may comprise nanometer-sized polymer latex particles. The nanometer-sized polymer latex particles may have a size/diameter/particle size/particle size distribution/average particle size in the range of from about 50 nm to about 1000 nm, from about 50 nm to about 500 nm, from about 50 nm to about 300 nm, from about 100 nm to about 300 nm. In various embodiments, the nanometer-sized polymer latex particles have a size/diameter/particle size/particle size distribution/average particle size of about 200 nm. A desired thickness of polymer layer over the primary capsule may be achieved by determining the size/diameter/volume of the polymer latex particles to be used. In various embodiments, as the method uses polymer particle dispersions to form the second polymer layer (i.e. the coating layer), various embodiments of the disclosed method have the advantageous ability to control layer thickness in one step. In various embodiments, the method does not require multiple depositions or surface modifications and thus may allow for greater control over the wall thickness of capsules by leveraging on the polymer latex size i.e. the wall thickness are less influenced by the molecular weight of polymers. In various embodiments, the organic polymer latex particle is substantially non-capsular.

In various embodiments, the organic polymer latex particles have a polydispersity index (PDI) of from about 0.005 to about 1, from about 0.005 to about 0.50, from about 0.005 to about 0.80, from about 0.010 to about 0.200, from about 0.015 to about 0.150, from about 0.020 to about 0.100, from about 0.025 to about 0.090 or from about 0.030 to about 0.080. In one example, the organic polymer latex particles may have a PDI of from about 0.005 to about 0.050 at about 40° C. and a PDI of from about 0.050 to about 0.100 at about 20° C. In another example, the organic polymer latex particles may have a PDI of from about 0.010 to about 0.050 at about 40° C. and a PDI of from about 0.050 to about 0.160 at about 20° C. In yet another example, the organic polymer latex particles may have a PDI of from about 0.010 to about 0.030 at about 40° C. and a PDI of from about 0.030 to about 0.180 at about 20° C. In still yet another example, organic polymer latex particles may have a PDI of from about 0.080 to about 1 at about 40° C. and a PDI of from about 0.800 to about 1 at about 20° C.

In various embodiments, the organic polymer latex particles are temperature responsive. For example, the organic polymer latex particles may exhibit a reversible decrease in size with an increase in temperature. For example, the organic polymer latex particles may decrease in size by no less than about 50% with an increase in temperature from about 20° C. to about 40° C.

In various embodiments, the method further comprises a step of forming the organic polymer latex particles by emulsion polymerization.

In various embodiments, the primary capsule comprises an inorganic capsule and the shell of the primary capsule comprises an inorganic shell. However, it should be appreciated that the primary capsule may comprise other different types of capsules (other than inorganic capsules). In particular, based on a desired application, the primary capsule may be chosen to be one that best suits that desired application. Accordingly, it should also be appreciated that embodiments of the methods disclosed herein are suitable for a wide variety of capsules and are not limited to inorganic capsules as the primary capsules. Without being bound by theory, it is believed that the high compatibility of embodiments of methods disclosed herein with a large variety of different types of primary capsules stems from the physical steps rather than chemical steps being employed to provide the polymer coating over the primary capsule shell.

In various embodiments, the at least one primary capsule comprises at least one inorganic particle selected from the group consisting of silica capsule, zirconia capsule, titania capsule or combinations thereof. Accordingly, in various embodiments, the hybrid/composite capsule comprises a silica-polymer capsule, a zirconia-polymer capsule or a titania-polymer capsule. The inorganic shell may be porous or mesoporous.

The at least one primary capsule may have a size/diameter/particle size/particle size distribution/average particle size in the range of from about 1 μm to about 100 μm, 10 μm to about 100 μm, from about 20 μm to about 100 μm, from about 30 μm to about 100 μm, from about 40 μm to about 100 μm, from about 50 μm to about 100 μm, from about 60 μm to about 100 μm, from about 70 μm to about 100 μm, or about 10 μm, about 20 μm, about 30 μm, about 40 μm, about 50 μm, about 60 μm, about 70 μm, about 80 μm, about 90 μm or about 100 μm.

In various embodiments, the method further comprises a step of adding one or more of a silica precursor, a zirconia precursor or a titania precursor to a template, optionally a substantially spherical template, in water/aqueous medium to form the at least one inorganic capsule. The silica precursor may comprise a tetraalkyl orthosilicate, a trialkoxyalkylsilane or a silicon alkoxide (alkoxy silane) that is optionally selected from the group consisting of tetramethyl orthosilicate (TMOS), tetraethyl orthosilicate (TEOS), tetrapropyl orthosilicate (TPOS), methyltrimethoxysilane (MTMS), methyltriethoxysilane (MTES) or the like or combinations thereof. The titania precursor may comprise titanium(IV) isopropoxide and titanium(IV) bis(ammonium lactato)dihydroxide or the like or combinations thereof. The zirconia precursor may comprise zirconium isopropoxide, zirconium etoxide, zirconium n-butoxide or the like or combinations thereof.

The method may further comprise a step of growing a shell of the primary capsule such as a silica shell, a zirconia shell or a titania shell on the template by a hydrolysis condensation reaction to form the at least one primary capsule (e.g. an inorganic capsule). In various embodiments, template comprises an emulsified droplet. The droplet may be substantially hydrophobic. The droplet may be micron- or submicron-sized. In various embodiments, the method does not comprise stabilizing the droplet by pickering emulsion for the purpose of forming a shell directly on the template droplet. Additionally, in various embodiments, the template does not comprise a monomer or an initiator. In various embodiments, the template does not comprise an ionic liquid.

In various embodiments, the droplet comprises an oil droplet, an alkane droplet or combinations thereof. The droplet may comprise oil, silicone oil, hydrocarbon, alkane, pentane, hexane, dodecane, low-boiling alkane, organic solvent, non-polar solvent, low-boiling organic solvent, active including sensitive active (e.g. sensitive to free radical reactions), aroma, aroma oil, flavour, fragrance, perfume, drug, therapeutic, cosmetic, skin care substance, consumer care substance or combinations or mixtures or derivatives thereof.

Accordingly, the method may further comprise a step of emulsifying one or more of the following to form the template: oil, silicone oil, hydrocarbon, alkane, pentane, hexane, dodecane, low-boiling alkane, organic solvent, non-polar solvent, low-boiling organic solvent, active including sensitive active (e.g. sensitive to free radical reactions), aroma, aroma oil, flavour, fragrance, perfume, drug, therapeutic, cosmetic, skin care substance, consumer care substance or combinations or mixtures or derivatives thereof.

In various embodiments, the method further comprises a step of providing/adding a surfactant to stabilize the template prior to the addition of a shell precursor e.g. one or more of a silica precursor, a zirconia precursor or a titania precursor. The surfactant may comprise one or more properties described herein.

In various embodiments, the method further comprises a step of evaporating the template from the at least one primary capsule to form a hollow capsule. The step of evaporating the template may comprise warming/heating the at least one primary capsule, optionally to a temperature that is close to the boiling point of the template. In various embodiments, evaporating the template comprises warming/heating the at least one primary capsule to a temperature that is no more than about 60° C., no more than about 55° C., no more than about 50° C., no more than about 45° C., no more than about 40° C., no more than about 39° C., no more than about 38° C., no more than about 37° C., no more than about 36° C., no more than about 35° C., no more than about 34° C., no more than about 33° C., no more than about 32° C., no more than about 31° C., no more than about 30° C., no more than about 29° C., no more than about 28° C., no more than about 27° C. or no more than about 26° C. In some embodiments, the template comprises a low boiling organic solvent. In one embodiment, the template comprises pentane.

In various embodiments, the method does not comprise adding/providing a vesicle template. For example, the method may not comprise adding/providing a polymer emulsion such as a PNIPAM emulsion particle as a template. According, in various embodiments, the method does not comprise growing a shell such as a silica shell around a PNIPAM emulsion particle template. Therefore, in various embodiments, the method does not comprise calcination or high temperatures to remove a template.

The methods disclosed herein may further comprise one or more post heterocoagulation steps. For example, the method may further comprise a step of purifying the hybrid/composite capsule formed in the mixture to remove any impurities such as excess/free polymer particles. The step of purifying the hybrid/composite capsule may comprise centrifuging the mixture, creaming the mixture, allowing the mixture to settle and subsequently decanting the mixture etc. Additional process engineering steps and automated additions or flow chemistry may also be introduced to avoid extra polymer particles without purification step.

The method may also further comprise a step of subjecting the purified hybrid/composite capsule to a temperature cycle to form a film.

The method may also further comprise a step of concentrating the hybrid/composite capsules to obtain a desirable concentration of the hybrid/composite capsules, optionally in the form of a colloidal dispersion in water. In various embodiments, the concentrated hybrid/composite capsules remain substantially intact i.e. no leakages or breakages.

The method may further comprise a step of drying the hybrid/composite capsules, optionally by freeze drying or spray drying the hybrid/composite capsules with appropriate protecting agents or additives, to produce solid forms of the hybrid/composite capsules. In various embodiments, the dried hybrid/composite capsules remain substantially intact i.e. no leakages or breakages.

The method may further comprise a step of loading one or more of the following into the hybrid/composite capsules: oil, silicone oil, active including sensitive active (e.g. sensitive to free radical reactions), aroma, aroma oil, flavour, fragrance, perfume, drug, therapeutic, cosmetic, skin care substance, consumer care substance or combinations or mixtures or derivatives thereof.

In various embodiments, the steps following the step of adding the at least one primary capsule to the polymer latex particles and preceding the purifying step are conducted as a one-pot synthesis/one-step direct synthesis process to produce the hybrid/composite capsule.

In various embodiments, the method has high encapsulation efficiency, high scalability and/or high reproducibility. For example, embodiments of the method may have a high encapsulation efficiency in the sense that (i) no less than about 40%, no less than about 35%, no less than about 30% or about 20-30% by weight of actives in the water/aqueous medium can be emulsified and encapsulated in the primary capsule and/or the hybrid/composite capsule with almost quantitative efficiency by use of embodiments of the method and/or (ii) leakage of the actives from the primary capsule and/or the hybrid/composite capsule is absent or minimal. The method may also have high scalability in the sense that the mixture can be scaled up to kilograms or even tons to produce a higher amount of the hybrid/composite capsules. Additionally, the method may have high reproducibility in the sense that the method may be performed reproducibly for no less than 5 times.

In various embodiments, the forming of the polymer layer/film over the primary capsule consist essentially of or consist of the heterocoagulation step. For example, the method does not comprise a plurality of (or multiple) deposition steps to repeatedly deposit polymer layers on the primary capsule to achieve a desired thickness of polymer layer over the primary capsule. In various embodiments, the method does not require additional deposition step(s) after the heterocoagulating step.

There is also provided a hybrid/composite capsule produced by the method disclosed herein. The hybrid/composite may therefore have one or more properties previously described.

In various embodiments, the hybrid/composite capsule comprises a primary capsule having a shell; and a polymer coating layer over the shell of the primary capsule. The hybrid/composite capsule produced by the method disclosed herein may comprise a hollow core or one or more actives/substances encapsulated by a hybrid/composite shell.

In various embodiments, the thickness of the hybrid/composite shell is from about 20 nm to about 1100 nm, from about 25 nm to about 1050 nm, from about 30 nm to about 1000 nm, from about 35 nm to about 950 nm, from about 40 nm to about 900 nm, or no less than about 20 nm, no less than about 25 nm, no less than about 30 nm, no less than about 35 nm or no less than about 40 nm.

In various embodiments, the hybrid/composite shell is substantially non-porous or hermetically sealed. The shell of the primary capsule may be substantially hermetically sealed by the polymer coating layer. For example, the hybrid/composite shell may comprise organic polymer latex particles or derivatives thereof blocking one or more pores of a shell of the primary capsule. Advantageously, the capsule is capable of containing cargoes/actives without substantial undesirable leakage. Therefore, in various embodiments, the hybrid/composite capsule remains substantially intact/stable/non-brittle/devoid of rupture/leakage (i.e. does not break) under high vacuum, e.g. during SEM analysis or when subject to high mechanical forces/stress or high shear. For example, when the hybrid/composite capsule is substantially resistant to breaking under scanning electron microscopy (SEM) vacuum conditions, it will be evident from the SEM images of the capsule. Advantageously, the one or more cargoes present within the capsule may be beneficially isolated/protected from an external environment for example under ambient conditions such as when the capsule is in storage.

The hybrid/composite shell may comprise an inner layer substantially made up of an inorganic material optionally selected from one or more of silica, zirconia, titania and combinations thereof, and an outer layer that is substantially a polymer layer derived from organic polymer latex particles or derivatives thereof. In various embodiments, the organic polymer coating comprises poly-N-isopropyl acrylamide (PNIPAM), poly-methyl-methacrylate-co-poly-styrene-co-polyethyl-hexyl-acrylate-co-poly-acrylic acid, poly caprolactone (PCL), poly valerolactone, poly butyrolactone, polyurethane, polyamide, polyacrylic acid or combinations thereof and the at least one primary capsule comprises a silica capsule, a zirconia capsule, a titania capsule or combinations thereof.

In various embodiments, the outer layer is a direct coalesced form/product of polymer latex particles and is substantially similar in chemical composition with the polymer latex particles. In various embodiments, the hybrid/composite shell does not consist of double-layered silica shell.

The outer layer may be substantially uniform in thickness. The outer layer may comprise about 10%, about 15%, about 16%, about 17%, about 18%, about 19%, about 20%, about 21%, about 22%, about 23%, about 24%, about 25% or about 30% of the hybrid/composite capsule.

In various embodiments, the outer layer is physically deposited on the inner layer. In various embodiments, the hybrid/composite shell does not comprise a polyelectrolyte such as a poly(allylamine hydrochloride) (PAH). Accordingly, the hybrid/composite shell may be substantially inert e.g. does not react with an encapsulated active.

The hybrid/composite capsule may be substantially spherical in shape. The hybrid/composite capsule may be micron- or submicron-sized. In various embodiments, the hybrid/composite capsule has a size/diameter/particle size/particle size distribution/average particle size in the range of from about 100 nm to about 100 μm, from about 500 nm to about 100 μm, from about 1 μm to about 100 μm, 10 μm to about 100 μm, from about 20 μm to about 100 μm, from about 30 μm to about 100 μm, from about 40 μm to about 100 μm, from about 50 μm to about 100 μm, from about 60 μm to about 100 μm, from about 70 μm to about 100 μm, no less than about 70 μm, no less than about 60 μm, no less than about 50 μm or no less than about 40 μm.

In various embodiments, the hybrid/composite capsule comprises one or more actives loaded in a core of the capsule that is encapsulated by the shell of the primary capsule. For example, the hybrid/composite capsule may be loaded with one or more cargoes selected from the following: oil, silicone oil, active including sensitive active (e.g. sensitive to free radical reactions), aroma, aroma oil, flavour, fragrance, perfume, drug, therapeutic, cosmetic, skin care substance, consumer care substance, combinations or mixtures or derivatives thereof. In various embodiments, the hybrid/composite shell is substantially inert e.g. does not react with an encapsulated active.

In various embodiments, the hybrid/composite capsule has a loading capacity of from about 65% to about 95% by weight, from about 70% to about 90% by weight, from about 75% to about 85% by weight, or no less than about 95% by weight, no less than about 90% by weight, no less than about 85% by weight, no less than about 80% by weight, no less than about 75% by weight, no less than about 70% by weight or no less than about 65% by weight.

In various embodiments, the hybrid/composite capsule comprises one or more of the following properties: non-toxic, hypoallergenic, biocompatible, degradable, environmentally benign, chemically stable and physically stable. Therefore, in various embodiments, the capsules may be useful in an application selected from the group consisting of: coating, therapy, medicine, agriculture, catalyst, printing, film, fiber, cosmetics, cosmeceutical, consumer care, personal care, health care, stimuli-driven delivery, and combinations thereof.

In various embodiments, the capsule is capable of releasing at least a portion of the one or more cargoes when stimulated (e.g. the polymer outer layer of the hybrid/composite capsule is degradable under suitable conditions to expose pores on the primary capsule). For example, capsule may be capable of releasing at least a portion of the one or more cargoes when stimulated by a predetermined level or change in one or more of a salt concentration, a pH, a temperature or a mechanical pressure etc.

There is also provided a formulation or colloidal suspension/dispersion comprising a plurality of the hybrid/composite capsules disclosed herein. In various embodiments, the hybrid/composite capsules are substantially monodispersed. For example, the hybrid/composite capsules may have a substantially uniform size distribution. In various embodiments, the solid content of the formulation or colloidal suspension/dispersion is from about 0.010 g/mL to about 0.015 g/mL, or about 0.013 g/mL (or 0.014 g/g).

In various embodiments, the capsule/formulation is compatible for one or more of the following applications: perfumes, coatings, medicines (e.g. considered as in the “generally recognized as safe” (GRAS) category by United States Food and Drug Administration (FDA)), agricultural chemicals, catalysts, printings, films, fibers and cosmetics (e.g. with good skin feeling).

### BRIEF DESCRIPTION OF FIGURES

FIG. 1 is a schematic flowchart 100 for illustrating a method of preparing a hybrid capsule in accordance with various embodiments disclosed herein.

FIG. 2 is a schematic diagram 200 for illustrating a method of preparing a hybrid capsule in accordance with various embodiments disclosed herein.

FIG. 3 is a schematic diagram 300 of a hybrid capsule in accordance with an example embodiment disclosed herein.

FIGS. 4A and 4B are scanning electron microscopy (SEM) images of poly(N-isopropylacrylamide) (PNIPAM) polymer latex prepared via emulsion polymerization with 1% acrylic acid (AA) and 1% N,N′-Methylenebis(acrylamide) (BIS) crosslinker by weight of the monomer latex particles in the absence of a SDS surfactant, and dried at an elevated temperature (e.g. at 90° C.), in accordance with an example embodiment disclosed herein. FIG. 4A shows an SEM image taken at 5,000× magnification, with the scale bar representing 1 μm. FIG. 4B shows an SEM image taken at 10,000× magnification, with the scale bar representing 1 μm.

FIG. 5 is a dynamic light scattering Z-average particle size (DLS Z-ave) vs. temperature graph showing the temperature response of PNIPAM polymer latex synthesized for heterocoagulation studies, in accordance with an example embodiment disclosed herein. As shown, a higher temperature led to decreased particle size, thereby demonstrating the temperature responsiveness of the latex.

FIG. 6A is a dark field microscopic image of silica capsule dispersion in water and FIG. 6B is a dark field microscopic image of hetero-coagulated silica capsules with poly-NIPAM latex particles prepared from a method in accordance with an example embodiment disclosed herein. The scale bar in both figures represents 50 μm.

FIG. 7 is a graph showing the particle size distribution of a silica capsule obtained after heterocoagulation.

FIG. 8A is a thermogravimetric analysis (TGA) graph of silica capsules and FIG. 8B is a thermogravimetric analysis (TGA) graph of hetero-coagulated silica capsules with poly-NIPAM latex particles prepared from a method in accordance with an example embodiment disclosed herein.

FIGS. 9A-9C are scanning electron microscopy (SEM) images of sub-20 micron silica capsules stabilised by cetyltrimethylammonium bromide (CTAB) in accordance with an example embodiment disclosed herein, taken at various stages of polymer coating. These images are captured after 2-3 hours after heterocoagulation and film formation under SEM conditions. FIG. 9A shows an SEM image taken at 5,000× magnification, with the scale bar representing 1 μm. FIG. 9B shows an SEM image taken at 5,000× magnification, with the scale bar representing 1 μm. FIG. 9C shows an SEM image taken at 1,000× magnification, with the scale bar representing 10 μm.

FIG. 10A is a scanning electron microscopy (SEM) image of original silica capsules and FIG. 10B is a scanning electron microscopy (SEM) of polycaprolactone particles coated on silica capsules after hetero-coagulation in accordance with an example embodiment disclosed herein. FIG. 10A shows an SEM image taken at 1,000× magnification, with the scale bar representing 10 μm. FIG. 10B shows an SEM image taken at 2,000× magnification, with the scale bar representing 10 μm.

### EXAMPLES

Example embodiments of the disclosure will be better understood and readily apparent to one of ordinary skill in the art from the following examples, tables and if applicable, in conjunction with the figures. It should be appreciated that other modifications related to structural, and chemical changes may be made without deviating from the scope of the invention. Example embodiments are not necessarily mutually exclusive as some may be combined with one or more embodiments to form new example embodiments. The example embodiments should not be construed as limiting the scope of the disclosure.

### Example 1: Method of Preparing A Hybrid Capsule

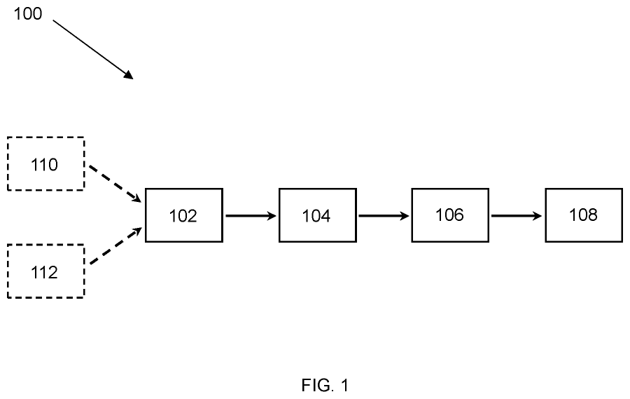

FIG. 1 is a schematic flowchart 100 for illustrating a method of preparing a hybrid capsule in accordance with various embodiments disclosed herein. At step 104, the organic polymer latex particles in a suspension/dispersion/mixture are heterocoagulated with at least one primary capsule such as an inorganic capsule so that an organic polymer film can be eventually formed over the shell of the primary capsule (e.g. inorganic shell).

It will be appreciated that in some embodiments, the method may also include steps before and after step 104. For example, prior to step 104, at least one primary capsule may be introduced into a volume of organic polymer latex particles to form the suspension/dispersion/mixture at step 102. Likewise, subsequent to step 104, the organic polymer latex particles may be further physically cured by using at least one of temperature or pH as a curing means to form an organic polymer coating layer over a shell of the primary capsule, thereby obtaining a hybrid capsule at step 106. In addition, at step 108, the hybrid capsule is further purified to remove any impurities such as excess/free polymer particles and/or further concentrated to obtain a desired concentration.

While in various embodiments, the method relies on the use of preformed organic polymer latex particles and primary capsules, it will also be appreciated that optional steps to prepare the polymer latex particles and primary capsules may also be incorporated in various embodiments of the method. For example, prior to step 102, the method may optionally comprise step 110 for synthesizing the primary capsule and may also optionally comprise step 112 for synthesizing the organic polymer latex particles. The dotted lines of boxes containing steps 110 and 112 emphasise that these steps may be absent in some embodiments of the present disclosure as the primary capsule(s) and/or organic polymer latex particles may be preformed or purchased/obtained commercially. For example, preformed silica capsules may be used as the primary capsule(s) and preformed (responsive) polymer latex may be used as the organic polymer latex.

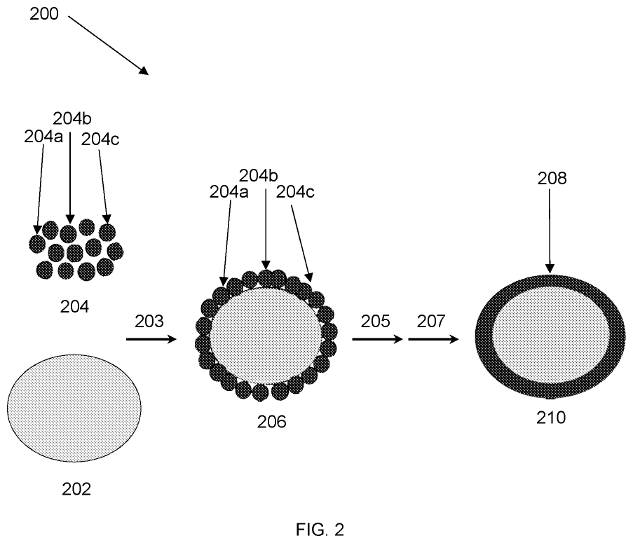

FIG. 2 is a schematic diagram 200 for illustrating a method of preparing a hybrid capsule in accordance with various embodiments disclosed herein. A primary capsule 202 is introduced into a volume of organic polymer latex particles 204a, 204b and 204c, collectively known as organic polymer latex 204 as shown by arrow 203 to form a suspension/dispersion/mixture 206. The organic polymer latex particles 204a, 204b and 204c then undergo heterocoagulation as shown by arrow 205 and physical curing as shown by arrow 207 to form an organic polymer coating layer 208 over a shell of the primary capsule 202, thereby forming a hybrid capsule 210. In other words, in various embodiments, the steps may include resuspension/dispersion of the polymer latex particles with the primary capsule followed by heterocoagulation such that fusion of the organic polymer latex particles will occur thereby forming film around the shell of the primary capsule. Thereafter, post processing steps such as purification and concentration of the capsules may take place. It will be appreciated that although not shown in the figure, the interior/core of the primary capsule 202 may be empty or may be filled/loaded with cargo such as active molecules.

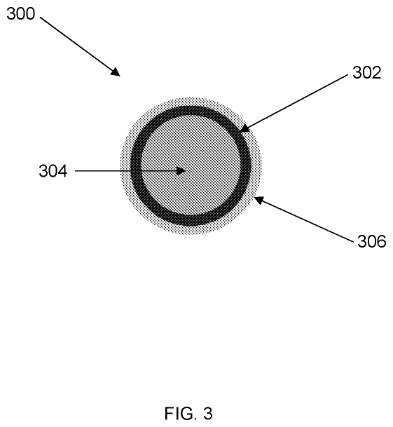

FIG. 3 is a schematic diagram of a hybrid capsule in accordance with various embodiments disclosed herein. The hybrid capsule 300 comprises a primary capsule having a shell 302 encapsulating an empty or a loaded/filled core 304 and an organic polymer coating layer 306. In this example, the hybrid capsule has a size/diameter/particle size/particle size distribution/average particle size in the range of from about 5 μm to about 50 μm.

### Example 2: Primary Capsule(s)

Prior to the synthesis of hybrid capsules/polymer reinforced structures, primary capsules stabilised by a surfactant were synthesized. In this example, the primary capsules synthesized were silica hollow microspheres stabilised by cetyltrimethylammonium bromide (CTAB) as the surfactant.

36.4 mg of CTAB was dissolved in 100 mL of deionised (DI) water in a 250 mL reaction vessel. The solution was stirred at 600 rpm for thirty minutes to ensure that all of the surfactant was dissolved. 10 mL of n-pentane was injected into the solution and was stirred for thirty minutes at 600 rpm. Before the addition of TEOS, light microscopy was taken to ensure that the particles have an average particle size of 30 μm. 4 mL of TEOS was added via a programmed syringe pump at 80 μL/min to the solution. After the addition of TEOS was completed, the stirring speed was lowered to 400 rpm. A sample of the solution was taken for analysis using brightfield microscopy. Microscopic images were recorded for four days. SEM analysis was done on day 4. The resultant silica capsules were post processed for other applications.

### Example 3: Organic Polymer Latex Particles

Prior to performing heterocoagulation of organic polymer latex particles with primary capsule(s), organic polymer latex particles were synthesized using emulsion polymerization. In this example, the organic polymer latex particles synthesized were poly(N-isopropylacrylamide) (PNIPAM) latex particles having an average particle size of about 200 nm.

150 mL of D.I. water was heated to 90° C. in the 1 L-jacketed reactor at the stirring speed of 200 rpm. 1.5 g of ammonium persulfate and 1.5 g of sodium hydrogen carbonate was added. 30 g of N-Isopropylacrylamide (NIPAM) and 0.3 g of N,N′-methylenebis (acrylamide) (BIS) dissolved in 750 mL of water was added into the reactor at the feed rate of 25 mL/min for 30 min. The mixture was stirred for another 30 min after the second feed of 0.3 g NIPAM, 0.03 g BIS and 0.3 g acrylic acid (AA) dissolved in 7.5 mL water was fed into the reactor at the rate of 1.5 mL/min. The total 900 mL mixture was allowed to stir at 90° C. for another 3 hr. The final latex was then filtered to remove aggregates and stored in glass bottle in 60° C. oven.

Latex particles were prepared in a few different batches to evaluate their stability against aggregation as well as their thermal response (i.e. response to temperature). The latex particles were characterized by scanning electron microscopy (SEM) and by performing dynamic light scattering (DLS) experiments.

### Scanning Electron Microscopy (SEM) of Latex Particles

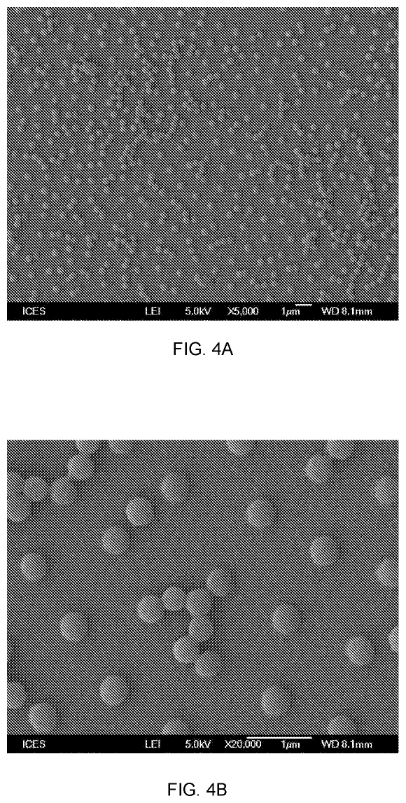

FIG. 4A and FIG. 4B are SEM images of PNIPAM latex particles taken at different degree of magnifications (i.e. 5,000× and 20,000× respectively). The latex particles were prepared via emulsion polymerization with 1% acrylic acid (AA) and 1% N,N′-methylenebis (acrylamide) (BIS) crosslinker by weight of the monomer latex particles in the absence of a SDS surfactant, and dried at an elevated temperature. As shown in FIG. 4A and FIG. 4B, the PNIPAM latex particles were stable against aggregation. The acrylic acid on the surface of the latex particles provided sufficient charge for latex particles to stabilize in aqueous system and prevented aggregation. Therefore, as demonstrated, a surfactant may not be required if an acid such as an acrylic acid is used as a monomer in the system.

### Dynamic Light Scattering (DLS) of Latex Particles

PNIPAM latex particles samples were prepared with varying concentrations of crosslinker (i.e. N,N′-methylenebis(acrylamide) (BIS)) and surfactant (i.e. sodium dodecyl sulfate (SDS)). A summary of the composition present (wt % based on the monomer latex particles) in the samples and their corresponding aggregation percentage (Aggr %) is provided in Table 1 below. The z-average particle size (DLS Z-Ave) and polydispersity index (PDI) results of the latex particles obtained from DLS experiments are also provided in Table 1. To test for reversibility in temperature response, the measurements were first taken at (i) a temperature of about 40° C., then at (ii) a temperature of about 20° C. and finally (iii) at a temperature of about 40° C. As shown in Table 1, the average particle size recorded at temperature (iii) for the samples remains relatively similar to those recorded at temperature (i). Therefore, the temperature response was proven to be reversible.

TABLE 1
Summary of composition and DLS results of PNIPAM samples
synthesized for hetero-coagulation studies

| | | | | Temperature | | | | | |
|---|---|---|---|---|---|---|---|---|---|
| | | | | 40° C. | | 20° C. | | 40° C. | |
| % (wrt to | BIS | SDS | Aggr | DLS Z- | | DLS Z- | | DLS Z- | |
| monomer) | (%) | (%) | (%) | ave (nm) | PDI | ave (nm) | PDI | ave (nm) | PDI |
| Sample A | 1 | 0.267 | 0 | 203.7 | 0.038 | 453.6 | 0.086 | 204.7 | 0.010 |
| (PNIPAM 3) | | | | | | | | | |
| Sample B | 0.2 | 0.267 | 0 | 207.2 | 0.014 | 494.6 | 0.144 | 209.6 | 0.031 |
| (PNIPAM 4) | | | | | | | | | |
| Sample C | 0 | 0.267 | 0 | 210.9 | 0.025 | 513.3 | 0.151 | 212.4 | 0.024 |
| (PNIPAM 5) | | | | | | | | | |
| Sample D | 1 | — | 48.1 | 553.5 | 0.104 | 20230.0 | 1.000 | 806.5 | 1.000 |
| (PNIPAM 6) | | | | | | | | | |

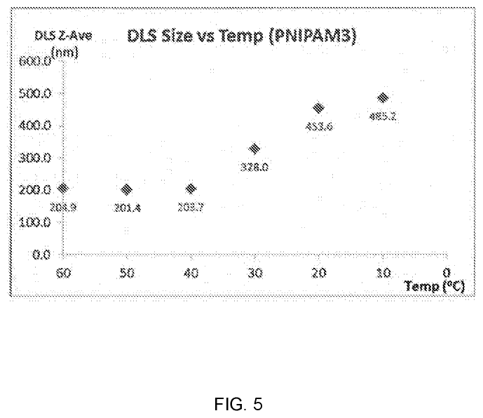

DLS measurements were also performed at varying temperatures from 10° C. to 60° C. and the results obtained on the average particle size of the PNIPAM samples are provided in FIG. 5. It is shown that a higher temperature led to decreased particle size (as expected), thereby demonstrating the temperature responsiveness of the latex particles.

### Example 4: Hetero-Coagulation of Organic Polymer Latex Particles (i.e. poly-NIPAM) with Primary Capsule(s)

In this example, hetero-coagulation experiments were carried out using poly-NIPAM latex particles as the organic polymer latex particles and CTAB stabilized silica capsules as the primary capsule(s).

Silica capsules dispersion 20.0 mL (10% w/w) was left to stand/allowed to cream in a sealed bottle over a day and then, the water dispersant was removed. The cream was then topped up with D.I. water and redispersed to the initial total volume of 20.0 mL. This process was repeated for another 2 cycles to remove free dissolved CTAB.

Under the working temperature of 60° C., the washed silica capsules dispersion was then added slowly over 1 min into 20.0 mL of PNIPAM latex dispersion (5% w/w) for heterocoagulation. For this step, the pH of the latex solution is alkaline (around 8.5) and pH of the silica capsule solution is acidic (around 3.2). Therefore, it will be appreciated that the pH of the heterocoagulation step can fluctuate between about 3 to about 9.5. The resulting mixture was then filtered and dispersed in 20.0 mL of D.I water.

The hybrid capsules obtained after the heterocoagulation step were also further purified. Purification was achieved by allowing the heterocoagulated, film formed hybrid capsules to cream, separating the aqueous layer containing no capsule from below, resuspending again in water by adding water and gentle stirring, stopping the stirring and allowing the hybrid capsules to cream again. This process was repeated 2-3 times to get pure heterocoagulated capsules with no free silica capsule or latex articles. Most of the personal care actives and perfume capsules are lower density and hence this method of creaming may work better for such lower density capsules.

For capsules that are denser than water, the capsules could be allowed to settle, and a similar procedure could be adopted by removing the top layer and repeating the washing steps.

### Dark Field Microscopy of (i) Silica Capsule and (ii) Hetero-Coagulated Silica Capsules With Poly-NIPAM Latex Particles

Evidence of temperature responsive poly-NIPAM latex particles adsorbing on CTAB stabilized silica capsules was demonstrated by careful optical microscopic analysis at various stages of the adsorption and poly-NIPAM film formation on the surface of the silica capsule.

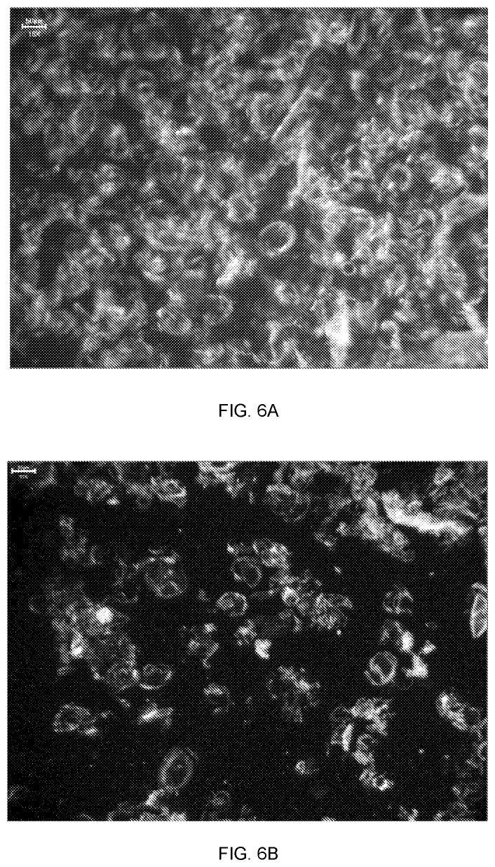

In the case of silica microspheres, SEM cannot be used as a characterization tool as microcapsules (e.g. having a size above 50 micrometer (>50 μm)) do not stay/remain stable under high vacuum conditions employed during measurement (e.g. SEM imaging) Therefore, the adsorption and film formation of poly-NIPAM latex particles on silica hollow spheres were demonstrated by dark field microscopy (see FIG. 6B).

FIG. 6A shows a dark field microscopic image of silica capsule dispersion in water. After heterocoagulation of poly-NIPAM latex particles with silica capsules, a dark field microscopic image was captured for the hetero-coagulated silica capsules with poly-NIPAM latex particles as shown in FIG. 6B. Particle size analysis of the hetero-coagulated silica capsules with poly-NIPAM latex particles using laser scattering shows a monomodal size distribution. This is indicative of a good nano-film formation of poly-NIPAM around the silica capsule structures.

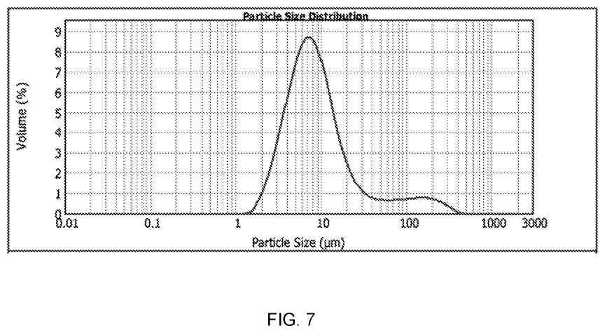

For the current measurement method, the refractive index of silica was used for determination of the particle size distribution. FIG. 7 shows the particle size distribution of a silica capsule obtained after heterocoagulation. Exact values of the particle size distribution of the heterocoagulated sample shown in FIG. 7 are provided in Table 2 below. A bimodal size distribution is shown, indicating some percentage of aggregation.

TABLE 2
Particle size distribution of a heterocoagulated
sample shown in FIG. 7

| Size | Volume |
|---|---|
| (μm) | in % |
| 0.100 | |
| 0.111 | 0.00 |
| 0.123 | 0.00 |
| 0.136 | 0.00 |
| 0.151 | 0.00 |
| 0.167 | 0.00 |
| 0.186 | 0.00 |
| 0.206 | 0.00 |
| 0.228 | 0.00 |
| 0.253 | 0.00 |
| 0.280 | 0.00 |
| 0.311 | 0.00 |
| 0.345 | 0.00 |
| 0.382 | 0.00 |
| 0.423 | 0.00 |
| 0.469 | 0.00 |
| 0.520 | 0.00 |
| 0.577 | 0.00 |
| 0.577 | |
| 0.640 | 0.00 |
| 0.709 | 0.00 |
| 0.786 | 0.00 |
| 0.871 | 0.00 |
| 0.966 | 0.00 |
| 1.071 | 0.00 |
| 1.187 | 0.00 |
| 1.316 | 0.00 |
| 1.459 | 0.01 |
| 1.617 | 0.06 |
| 1.793 | 0.25 |
| 1.988 | 0.50 |
| 2.204 | 0.76 |
| 2.443 | 1.10 |
| 2.708 | 1.51 |
| 3.002 | 1.97 |
| 3.328 | 2.49 |
| 3.328 | |
| 3.690 | 3.05 |
| 4.090 | 3.63 |
| 4.535 | 4.20 |
| 5.027 | 4.72 |
| 5.573 | 5.18 |
| 6.178 | 5.54 |
| 6.849 | 5.78 |
| 7.593 | 5.87 |
| 8.417 | 5.80 |
| 9.331 | 5.56 |
| 10.344 | 5.22 |
| 11.468 | 4.74 |
| 12.713 | 4.19 |
| 14.093 | 3.59 |
| 15.624 | 3.01 |
| 17.321 | 2.49 |
| 19.201 | 2.05 |
| 19.201 | |
| 21.286 | 1.66 |
| 23.598 | 1.37 |
| 26.160 | 1.11 |
| 29.001 | 0.90 |
| 32.151 | 0.75 |
| 35.642 | 0.63 |
| 39.512 | 0.55 |
| 43.803 | 0.50 |
| 48.559 | 0.47 |
| 53.832 | 0.46 |
| 59.678 | 0.45 |
| 66.159 | 0.45 |
| 73.343 | 0.45 |
| 81.307 | 0.46 |
| 90.136 | 0.46 |
| 99.924 | 0.47 |
| 110.775 | 0.49 |
| 110.775 | |
| 122.804 | 0.50 |
| 136.140 | 0.51 |
| 150.923 | 0.52 |
| 167.312 | 0.53 |
| 185.481 | 0.52 |
| 205.622 | 0.50 |
| 227.951 | 0.47 |
| 252.704 | 0.42 |
| 280.145 | 0.36 |
| 310.566 | 0.29 |
| 344.291 | 0.22 |
| 381.678 | 0.13 |
| 423.125 | 0.06 |
| 469.072 | 0.01 |
| 520.009 | 0.00 |
| 576.477 | 0.00 |
| 639.077 | 0.00 |
| 639.077 | |
| 708.475 | 0.00 |
| 785.406 | 0.00 |
| 870.696 | 0.00 |
| 965.246 | 0.00 |
| 1070.062 | 0.00 |
| 1186.261 | 0.00 |
| 1315.078 | 0.00 |
| 1457.883 | 0.00 |
| 1616.196 | 0.00 |
| 1791.700 | 0.00 |
| 1986.251 | 0.00 |
| 2201.951 | 0.00 |
| 2441.062 | 0.00 |
| 2706.139 | 0.00 |
| 3000.000 | 0.00 |

### Gravimetric Analysis and Thermogravimetric Analysis of (i) Silica Capsule and (ii) Hetero-Coagulated Silica Capsules With Poly-NIPAM Latex Particles

There are many ways to utilize the hetero-coagulated silica capsules with poly-NIPAM latex particles and one (ideal) way is to use the material in the form of dispersion in formulations. In this example, the solid content of the material (estimated gravimetrically) is 0.013 g/mL (or 0.014 g/g).

To determine the content of poly-NIPAM latex layer adsorbed on the silica capsules, thermogravimetric analysis (TGA) was performed on both (i) silica capsule and (ii) hetero-coagulated silica capsules with poly-NIPAM latex particles.

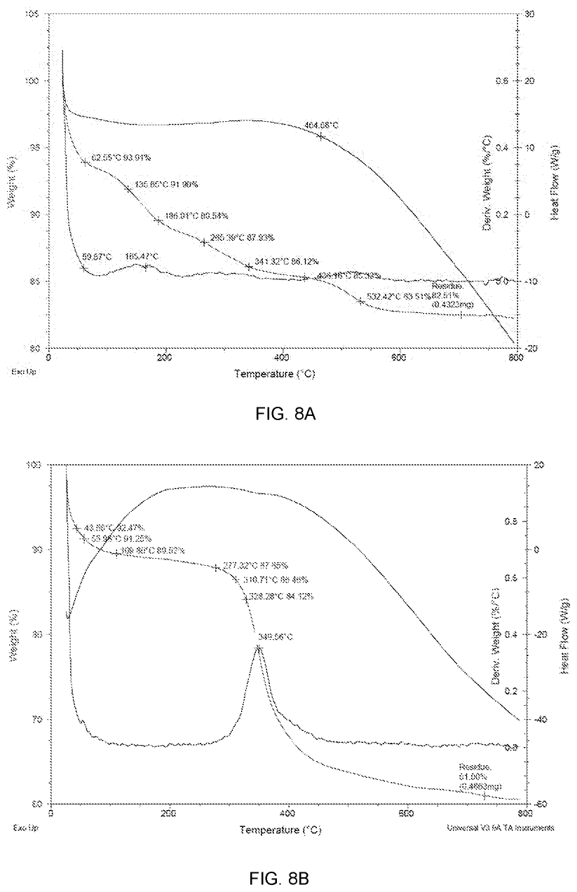

In FIG. 8A, the TGA result of silica micro capsules shows that the residual amount is 82.5%. After hetero-coagulation of poly-NIPAM latex particles with silica capsules, it is shown in FIG. 8B that the residual amount decreased to 61%. Therefore, this indicated that the content of poly-NIPAM latex adsorbed on the silica capsules is approximately 20%.

Analysis of the silica capsules vs. poly-NIPAM coated silica capsules using dark field microscopy and TGA proved that the hetero-coagulated silica capsules with poly-NIPAM latex particles synthesized in the examples are spherical micro-capsules having uniform size distribution with approximately 20% temperature responsive poly-NIPAM coating. Poly-NIPAM is also reported to be salt-responsive.

### Scanning Electron Microscopy of Sub-20 Micron Hetero-Coagulated Silica Capsules With Poly-NIPAM Latex Particles

To further prove that the hetero-coagulation was successfully achieved, the inventors performed synthesis of sub-20 micron silica capsules using CTAB as the surfactant, which are not breakable under SEM, i.e. able to withstand the vacuum condition used for SEM imaging

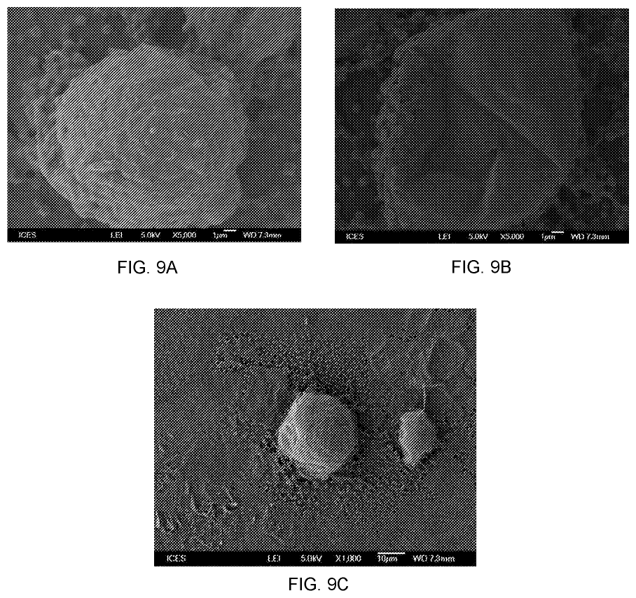

SEM images were obtained at various stages of the polymer coating process (see FIG. 9A, FIG. 9B and FIG. 9C). Based on the SEM images, it is shown very clearly that the inventors were able to coat a uniform layer of polymer around the silica capsules.

### Example 5: Hetero-Coagulation of Organic Polymer Latex Particles (i.e. Polycaprolactone) with Primary Capsule(s)

In this example, hetero-coagulation experiments were carried out using polycaprolactone particles as the organic polymer latex particles and silica capsules as the primary capsule(s).

Silica capsules dispersion 20.0 mL (10% w/w) was left to stand/allowed to cream in a sealed bottle over a day and then, the water dispersant was removed. The cream was then topped up with D.I. water and redispersed to the initial total volume of 20.0 mL. This process was repeated for another 2 cycles to remove free dissolved CTAB.

The washed silica capsules dispersion was then added slowly over 1 min into 20.0 mL of PCL latex dispersion (5% w/w), which may be purchased or made using conventional techniques such as that described in “Nano-emulsion formulation using spontaneous emulsification: solvent, oil and surfactant optimisation”, Fessi, H. et al. Int. J. Pharm. 2004, 280(1-2), 241-251”, the contents of which are fully incorporated by reference. The resulting mixture was then heated at 70° C. for 10 min with stirring and cooled to room temperature. For this step, the pH of the latex solution is alkaline (around 8.5) and pH of the silica capsule solution is acidic (around 3.2). Therefore, it will be appreciated that the pH of the heterocoagulation step can fluctuate between about 3 to about 9.5. It was then filtered and dispersed in 20.0 mL of D.I water.

The hybrid capsules were also purified using similar methods that were described in Example 4.

### Scanning Electron Microscopy of (i) Silica Capsule and (ii) Hetero-Coagulated Silica Capsules With Polycaprolactone Particles

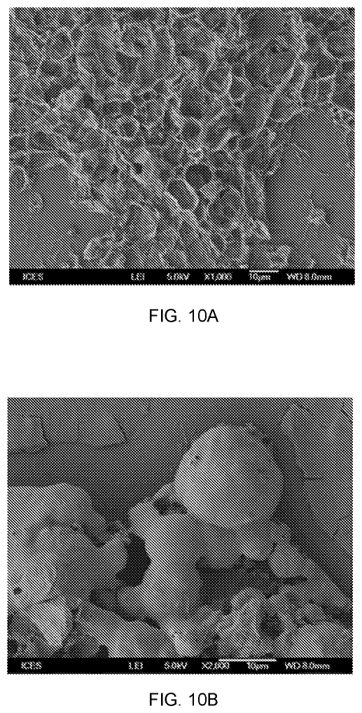

FIG. 10A shows the SEM image of original silica capsules. The silica capsules were fragile and break under vacuum conditions used for SEM imaging Therefore, only broken films were observed.

On the other hand, the SEM image of hetero-coagulated silica capsules with polycaprolactone particles (FIG. 10B) shows that the hybrid capsule did not break. As shown, capsules with spherical morphology were observed under SEM conditions, which is indicative of the robustness and stability of the hybrid capsule.

The examples above demonstrate the successful synthesis of a hybrid capsule by heterocoagulating organic polymer latex particles with a primary capsule. The examples also show that stability of the capsules is enhanced by having an additional layer of polymer around the micron sized capsules such as silica capsules. The results therefore show that the common drawbacks of micron sized capsules (such as capsule breakability and porosity etc.) can be addressed by embodiments of the method disclosed herein.

### APPLICATIONS

Embodiments disclosed herein provide a method that involves applying a heterocoagulation approach and creating a polymer film to impart stability to primary capsules such as silica capsules with actives (which normally have lot of instability issues such as capsule breakability, settling or creaming in water depending on the density of the active).

The inventors have surprisingly found out that desirable capsules may be produced via a simple heterocoagulation approach to obtain a film of polymer on a shell of a primary capsule such as a silica shell without using any chemical modification, notwithstanding that the primary capsule used may be considered one that is originally unstable (e.g. fragile inorganic capsules such as silica capsules). This is surprising as there was no prior knowledge or indication that such a film coating is possible on a primary capsule surface to reinforce a primary capsule shell, without breaking the micron or submicron sized primary capsule.

Embodiments of the method are capable of producing hollow capsules (contrast with solid nanoparticles) that are biocompatible, degradable, environmentally benign, chemically and physically stable. For example, hybrid capsules produced by embodiments of the methods disclosed herein are advantageously stable under high vacuum such as the vacuum conditions used for SEM measurements.

Advantageously, embodiments of method involve the deposition of a polymer film on a primary capsule such as a silica capsule using polymer particles, particularly polymer latex particles, without a crosslinking chemistry or curing agent or organic solvent. For example, embodiments of the methods disclosed herein do not involve free radical chemistry which limits application potential and do not require a chemical reaction such as polymer grafting or surface functionalization (such as surface initiated ATRP) of a silica capsule. As such, embodiments of the methods disclosed herein do not need any block co-polymer (which is more expensive and require synthetically more steps to produce as compared to the polymer latex particles used in embodiments of the method) or controlled radical polymerization. Special silane monomers such as organo alkoxy silanes to render the silica surface hydrophobic are also not required in embodiments of the methods disclosed herein.

In this regard, as embodiments of the methods disclosed herein do not use coupling chemistry and are based on physical phenomena, sensitive actives can be encapsulated and applied. Advantageously, embodiments, of the methods disclosed herein are suitable encapsulating a wide variety of active compounds, oils, hydrophobic actives/formulations and also for sensitive actives e.g. actives that react with free radicals. As such, embodiments of the methods disclosed herein are suitable for cosmetic/consumer-based applications such as for perfumes or aromatic oils or other consumer-based product encapsulation.

As compared to chemical-based methods, embodiments of the disclosed method rely primarily on water-based technology without the need to use organic solvents. For example, water-based chemistry may be employed for both silica capsule and latex synthesis. Advantageously, the water-dispersed polymer latex used do not require chemical curing but instead rely on physical curing (e.g. by making use of temperature or pH) of a polymer layer on a primary capsule shell such as silica shell. The water-based polymer latex nanoparticles may then be heterocoagulated on a primary capsule e.g. silica capsule surface and allowing polymer chains to interpenetrate to seal the pores and create a polymer shell on top of the silica hollow sphere. As such, embodiments of the methods disclosed herein are capable of blocking porous channels in primary capsules by coating polymer leading to longer and better protection of the encapsulated material. The hybrid capsules may thus be hermetically sealed unlike in the case of pure silica capsules.

Embodiments of the methods disclosed herein also versatile. For example, embodiments of the methods disclosed herein are compatible with polymers having favorable film forming properties and wide-ranging polydispersity indexes (including degradable/biodegradable, bioresorbable and/or non-toxic polymers). In one particular example, embodiments of the disclosed method are capable of coating polycaprolactone latex around otherwise breakable silica capsules. Given that polycaprolactone is biodegradable, bioresorbable and non-toxic, cosmetic and even cosmeceutical applications become easy with this approach.

In embodiments of the disclosed method, the polymer can be varied to generate different properties including stimuli responsive and degradable nature. Embodiments of the method disclosed herein make it possible to introduce multiple functionalities to a capsule easily which allows the method to be adapted as one that has a modular approach. This is possible as embodiments of the disclosed methods do not involve stabilization of droplets by pickering emulsion and thus allow for a variety of functionalities to be possible to be present on the capsule through the ability to use different polymers (including stimuli responsive polymers). As such, the capsules produced by embodiments of the methods disclosed herein may be customised to release their actives at certain predetermined conditions. The benefits offered by embodiments of the disclosed methods in terms of protection of the active and versatility of the release options, make the approach valuable.

Additionally, embodiments of the methods disclosed herein are also versatile in that they are compatible with primary capsules with or without the structural feature of a mesoporous shell and have the flexibility to work with both micron to submicron sized capsules to accordingly produce micron and submicron particles (e.g. from micro-sized to nano-sized capsule products) with relative ease. Advantageously, embodiments of the method are able to address or ameliorate the challenges faced by primary capsules such as silica microcapsules having nano pores and which typically have difficulty keeping small molecules (like perfume) actives inside the capsule. Advantageously, embodiments of the disclosed method are also able to produce reinforced/strengthened capsules over 50 μM, which are typically brittle and easily break when produced by conventional methods.

Advantageously, embodiments of the disclosed methods employ physical methods that are simple and scalable. Embodiments of the disclosed methods do not involve coacervation to create a polymer shell around a primary capsule such as a silica capsule to produce a hybrid capsule and do not require calcination to remove a template. Instead, embodiments of the methods disclosed herein use a single polymer latex (and not multiple polymers that is e.g. used in layer by layer polymer assembly around a particle). As this is unlike a layer-by-layer approach which requires several different layers to get the desirable thickness, embodiments of the method can achieve the desirable thickness in one single step and are easily scalable. Accordingly, embodiments of the disclosed method are simpler as compared to complex approaches using hydrogen bonding and layer by layer assembly. In addition, embodiments of the disclosed methods can be scaled up to kilograms and even tons since embodiments of the method depend mainly on shear force for breakdown of actives into droplets and surfactants such as cationic surfactants for stabilization of the droplets.

Furthermore, embodiments of the disclosed methods do not involve the more complex and less scalable steps of stabilization of droplets by pickering emulsion and then subsequently growing shell on the pickering stabilized emulsion to obtain hollow structures. Instead, embodiments of the disclosed methods involve creating a primary inner core such as an inorganic inner core, for example, a silica inner core, then producing a pickering emulsion using polymer particles, and then curing it on the surface to create a polymer shell, thus obtaining a hybrid capsule by simple steps.

As compared to known methods that involve in situ polymerization (which can be sort of classified as precipitation polymerization), embodiments of the disclosed method are able to use preformed commercially available functional polymers. As such, embodiments of the disclosed method are able to separate a polymer coating incorporation step from a primary capsule shell (e.g. silica) formation step, thus having more versatility and do not suffer from the shortcomings of an active reacting with the functional polymer. If desired, embodiments of the methods disclosed herein may optionally involve a polymerization step that is done separate in water to form latex particles which are adsorbed onto and cured to form a polymer shell around existing primary capsule shell such as silica shell. This allows a sufficiently distinct polymer layer around a first primary capsule shell such as a first silica shell to be formed and no cross-linking (chemical reaction) is involved in creating a second polymer shell/coating and hence only a physical approach is used for shell formation.

Advantageously, embodiments of the disclosed methods are capable of preparing an active loaded primary capsule e.g. silica capsule (as opposed to hard solid particle) whilst forming substantially uniform coating around the surface e.g. a silica surface using polymer latex particles (e.g. a reinforced silica shell by physical deposition of preformed polymeric nanoparticles on micron sized silica particles may be obtained). Accordingly, embodiments of the methods disclosed herein are capable of producing capsules with potentially higher stability to high shear compared to capsules in the art.

Embodiments of the disclosed methods also have high encapsulation efficiency since 20-30 wt % of actives in water can be emulsified and encapsulated in a primary capsule such as silica capsule with almost quantitative efficiency. Upon polymer coating, leakage of the encapsulated actives is substantially prevented and hence the efficiency is retained high.

Even more advantageously, embodiments of the disclosed methods have high reproducibility (the inventors have performed embodiments of the method reproducibly for more than 5 times in lab scale (1 L total volume)).

It will be appreciated by a person skilled in the art that other variations and/or modifications may be made to the embodiments disclosed herein without departing from the spirit or scope of the disclosure as broadly described. For example, in the description herein, features of different example embodiments may be mixed, combined, interchanged, incorporated, adopted, modified, included etc. or the like across different example embodiments. The present embodiments are, therefore, to be considered in all respects to be illustrative and not restrictive.

1. A method of preparing a hybrid capsule, the method comprising:
heterocoagulating organic polymer latex particles with a primary capsule to form an organic polymer coating layer over a shell of the primary capsule.

2. The method of claim 1, wherein the hybrid capsule is an organic-inorganic capsule and the primary capsule is an inorganic capsule.

3. The method of claim 1, of wherein the heterocoagulating step is at least partly carried out at a temperature that is no less than the glass transition temperature (T_{g}) of the organic polymer latex particles.

4. The method of claim 3, wherein the heterocoagulating step is at least partly carried out at a temperature of from 10° C. to 80° C. and/or at a pH from 2 to 11.

5. The method of claim 1, wherein the heterocoagulating step is carried out in the presence of two opposing charges, a first charge being associated with the organic polymer latex particles and a second charge being associated with the primary capsule, the second charge having a polarity that is opposite to that of the first charge.

6. The method of claim 1, further comprising introducing the primary capsule into a larger volume of the polymer latex particles prior to the heterocoagulating step.

7. The method of claim 6, wherein the step of introducing the primary capsule comprises introducing a plurality of primary capsules until the concentration of the primary capsules in the mixture of primary capsules and organic polymer latex is from 10% to 40% by weight of the entire mixture.

8. The method of claim 1, wherein the heterocoagulating step to form a polymer coating layer over a shell of the primary capsule is substantially devoid of a polymerization reaction and, optionally, wherein the heterocoagulating step is carried out in the absence of an organic solvent.

9. (canceled)

10. The method of claim 1, wherein the organic polymer latex particles comprises poly-N-isopropyl acrylamide (PNIPAM) latex particles, poly-methyl-methacrylate-co-poly-styrene-co-polyethyl-hexyl-acrylate-co-poly-acrylic acid latex particles, poly caprolactone (PCL) latex particles, poly valerolactone latex particles, poly butyrolactone latex particles, polyurethane latex particles, polyamide latex particles, polyacrylic acid-containing latex particles or combinations thereof.

11. The method of claim 1, wherein the primary capsule comprises a silica capsule, a zirconia capsule, a titania capsule or combinations thereof.

12. The method of claim 1, wherein the polymer latex particles have an average particle size in the range of from 50 nm to 1000 nm and the primary capsule has an average particle size in the range of from 1 μm to 100 μm.

13. The method of claim 5, wherein the heterocoagulating step is carried out in the presence of at least two different surfactants comprising at least one cationic surfactant and at least one anionic surfactant and, optionally, wherein the at least two different surfactants are independently selected from the group consisting of a primary amine surfactant, a secondary amine surfactant, a tertiary amine surfactant, a quaternary amine surfactant, cetyl trimethylammonium bromide (CTAB), sodium dodecyl sulfate (SDS), carboxylic acid salt, sulfonic acid salt, phosphoric acid ester, alcohol sulfate, alkylbenzene sulfonate, and a combination thereof.

14. (canceled)

15. A hybrid capsule comprising:
a primary capsule having a shell; and
an organic polymer coating layer over the shell of the primary capsule.

16. The hybrid capsule of claim 15, wherein the hybrid capsule is an organic-inorganic capsule and the primary capsule is an inorganic capsule.

17. The hybrid capsule of claim 15, wherein the shell of the primary capsule is substantially hermetically sealed by the polymer coating layer.

18. The hybrid capsule of claim 15, wherein the hybrid capsule is micron- or submicron-sized.

19. The hybrid capsule of claim 15, wherein the hybrid capsule is substantially resistant to breaking under scanning electron microscopy (SEM) vacuum conditions.

20. The hybrid capsule of claim 15, wherein the hybrid capsule comprises one or more actives loaded in a core of the primary capsule that is encapsulated by the shell of the primary capsule.

21. The hybrid capsule of claim 15, wherein the organic polymer coating comprises poly-N-isopropyl acrylamide (PNIPAM), poly-methyl-methacrylate-co-poly-styrene-co-polyethyl-hexyl-acrylate-co-poly-acrylic acid, poly caprolactone (PCL), poly valerolactone, poly butyrolactone, polyurethane, polyamide, polyacrylic acid or combinations thereof and the primary capsule comprises a silica capsule, a zirconia capsule, a titania capsule or combinations thereof.

22. The hybrid capsule of claim 21, wherein the hybrid capsule is configured to release at least a portion of one or more actives from the core when stimulated by a change in one or more of a salt concentration, a pH, a temperature or a mechanical pressure.
